(19) United States
(12) Patent Application Publication
(10) Pub. No.: US 2023/0030491 A1
(43) Pub. Date: Feb. 2, 2023

(54) EMULSIFYING FORMULATIONS OF CANNABINOIDS AND/OR CANNABINOID EXTRACTS

(71) Applicant: OrganiGram Inc., Moncton (CA)

(72) Inventors: Jiahua Zhou, Moncton (CA); Zachary LeBlanc, Riverview (CA); Jeremie Doiron, Moncton (CA)

(21) Appl. No.: 17/787,472

(22) PCT Filed: Dec. 18, 2020
(86) PCT No.: PCT/CA2020/051766

§ 371 Date: Jun. 20, 2022

Related U.S. Application Data
(60) Provisional application No. 62/951,222, filed on Dec. 20, 2019.

(51) Int. Cl. A23L 33/105; A23L 29/10; A23L 2/52; A61K 36/185

(52) U.S. Cl. CPC A23L 33/105; A23L 29/10; A23L 2/52; A61K 36/185

(57) ABSTRACT

The present application includes liquid and solid emulsifying formulations, for example, self-emulsifying formulations of cannabinoids and/or cannabinoid extracts including a surfactant and optionally one or more oils. The present application also includes beverage products comprising the liquid or solid emulsifying formulations. Methods of preparing the liquid and solid emulsifying formulations are also disclosed.

### RELATED APPLICATIONS

The present application claims the benefit of priority of co-pending U.S. provisional patent application No. 62/951,222 filed on Dec. 20, 2019, the contents of which is incorporated herein by reference in its entirety.

### FIELD

The present application relates to emulsifying drug delivery formulations, for example, self-emulsifying drug delivery formulations and to processes for their preparation. More specifically, the application relates to liquid and solid emulsifying formulations of cannabinoids and/or cannabinoid extracts and uses thereof, for example, in food and beverage products.

### BACKGROUND

Cannabinoids are naturally lipophilic compounds that are practically insoluble in water. When ingested alone, they do not stimulate the release of digestive enzyme and bile, and therefore have very low bioavailability and long onset time. Excipient/carrier oils like triglycerides may be added to improve both the bioavailability and absorption of lipophilic active pharmaceutical ingredients (APIs). Upon digestion, they contribute to the micellization of lipophilic APIs and their subsequent absorption mediated by free fatty acids and monoglycerides. However, when cannabinoids are consumed together with oil, the onset time is limited by the digestion of oil, which is the prerequisite of micellization. The digestion and absorption rate of oil and oil-soluble APIs can be increased by lowering the size of the oil droplets with the help of emulsifiers, transforming large oil droplets into micro- or nano-emulsions. Emulsification through sonication, high-pressure homogenization, microfluidization, colloid milling, or other technologies has been used in industry for this purpose. The liquid emulsion can be further processed to solid with drying technology and the resultant solid emulsion can be reconstituted to liquid emulsions by dispersing in water.

A major drawback of these technologies is the cost and complexity of equipment and handling needed to manufacture consistent large scale lots of liquid or dried emulsions. If parameters of emulsification are not perfectly tuned the resultant emulsion will not be of sufficiently small size to be rapidly absorbed through the digestive tract, resulting in sub-optimal pharmacokinetic profiles. Further, if drying parameters are not perfectly tuned the resulting particle size and dissolving speed can be unpredictable or undesirable, generating a product that is, for example, dusty and difficult to dissolve and handle. Additionally, the resulting emulsions could be easily destroyed by heat, freeze-thaw cycle, and long-term storage. The process of providing an emulsifying formulation is generally complicated, with a high probability of error. Thus, there is a need for improved formulations having improved dissolution, stability, absorption and/or bioavailability.

### SUMMARY

The application relates to emulsifying drug-delivery formulations of cannabinoids. In an embodiment, the application relates to emulsifying drug-delivery formulations of cannabinoids which disperse in aqueous media to form emulsions.

Accordingly, the present application includes a liquid emulsifying formulation comprising:
i) one or more cannabinoids, and/or a cannabinoid extract; and
ii) one or more surfactants;
wherein the weight ratio of the one or more surfactants to the one or more cannabinoids, and/or the cannabinoid extract is from about 1:5 to about 100:1.

In an embodiment, the emulsifying formulation optionally further comprises one or more oils. Accordingly, the present application also includes a liquid emulsifying formulation comprising:
i) one or more cannabinoids, and/or a cannabinoid extract;
ii) one or more surfactants; and
iii) one or more oils,
wherein the weight ratio of the one or more surfactants to the one or more cannabinoids, and/or the cannabinoid extract is from about 1:5 to about 100:1, and the weight ratio of the one or more oils to the one or more cannabinoids, and/or the cannabinoid extract is from about 1:99.9 to about 99.9:1.

In an embodiment, the liquid emulsifying formulation is converted into a solid emulsifying formulation using one or more water soluble carrier solid materials. Accordingly, the present application also includes a solid emulsifying formulation comprising:
i) one or more cannabinoids, and/or a cannabinoid extract;
ii) one or more surfactants; and
iii) one or more water soluble carrier solid materials,
wherein the weight ratio of the one or more surfactants to the one or more cannabinoids, and/or the cannabinoid extract is from about 1:5 to about 100:1.

In an embodiment, the solid emulsifying formulation further comprises one or more oils. Accordingly, the present application as includes a solid emulsifying formulation comprising:
i) one or more cannabinoids, and/or a cannabinoid extract;
ii) one or more surfactants;
iii) one or more oils, and
iv) one or more water soluble carrier solid materials,
wherein the weight ratio of the one or more surfactants to the one or more cannabinoids, and/or the cannabinoid extract is from about 1:5 to about 100:1, and the weight ratio of the one or more oils to the one or more cannabinoids, and/or the cannabinoid extract is from about 1:99.9 to about 99.9:1.

The present application also includes a solid emulsifying formulation comprising:
i) one or more cannabinoids, and/or a cannabinoid extract;
ii) one or more surfactants selected polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof;
iii) one or more oils selected from medium chain triglyceride (MCT) oils and long chain triglyceride (LCT) oils, and combinations thereof; and
iv) one or more water soluble carrier solid materials selected from one or more sugars, sugar alcohols, oligosaccharides, and polysaccharides, and combinations thereof,
wherein the weight ratio of the one or more surfactants to the one or more cannabinoids, and/or the cannabinoid extract is from about 1:5 to about 100:1; and the weight ratio of the one or more oils to the one or more cannabinoids, and/or the cannabinoid extract is from about 1:99.9 to about 99.9:1.

In an embodiment, the emulsifying formulation or solid emulsifying formulation further comprise one or more co-surfactants. In an embodiment, the weight ratio of the one or more co-surfactants to the one or more surfactants is from about 1:99.9 to about 99.9:1.

The present application also includes a method of preparing an emulsifying formulation comprising:

a) mixing one or more cannabinoids and/or a cannabinoid extract and one or more oils under conditions for producing an oil phase;

b) optionally, warming the oil phase;

c) combining one or more surfactants and optionally one or more co-surfactants with the oil phase under conditions for producing the emulsifying formulation.

In an embodiment, the method further comprises adsorbing the liquid emulsifying formulation onto the one or more water soluble carrier solid materials under conditions to produce the solid emulsifying formulation.

The present application also includes a liquid beverage product comprising the emulsifying formulations of the applications and an aqueous medium.

The present application also includes an additive for a food or beverage product wherein the additive comprises the emulsifying formulations of the application.

Accordingly, the present application also includes a composition comprising the emulsifying formulations of the application. In an embodiment, the composition is beverage product. In an embodiment, the composition is an edible consumable, a nutraceutical, a medicine, or pharmaceutical composition.

In an embodiment, the present application comprises, consists or consists essentially of the stated features, elements, components, groups, integers, and/or steps.

Other features and advantages of the present application will become apparent from the following detailed description. However, it should be understood that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

I. Definitions

Unless otherwise indicated, the definitions and embodiments described in this and other sections are intended to be applicable to all embodiments and aspects of the present application herein described for which they are suitable as would be understood by a person skilled in the art.

The term “emulsifying formulations of the application” and the like as used herein refers to the liquid and solid emulsifying formulations of the application.

The present application refers to a number of chemical terms and abbreviations used by those skilled in the art. Nevertheless, definitions of selected terms are provided for clarity and consistency.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps. As used herein, the word “consisting” and its derivatives, are intended to be close ended terms that specify the presence of stated features, elements, components, groups, integers, and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “a cannabinoid extract” should be understood to present certain aspects with a cannabinoid extract or two or more cannabinoid extracts. In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “effective amount” or “therapeutically effective amount” of a compound of the present disclosure is a quantity sufficient to, when administered to the subject, effect beneficial or desired results, including clinical results. The amount of a given compound of the present disclosure that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. “Treating” and “treatment” as used herein also include prophylactic treatment. Treatment methods comprise administering to a subject a therapeutically effective amount of one or more of the compounds of the application and optionally consist of a single administration, or alliteratively comprise a series of administrations. Treatment methods comprise administering to a subject a therapeutically effective amount of one or more of the formulations and compositions of the application and optionally consist of a single administration, or alliteratively comprise a series of administrations.

Palliating” a disease, disorder or condition means that the extent and/or undesirable clinical manifestations of a disease, disorder or condition are lessened and/or time course of the progression is slowed or lengthened, as compared to not treating the disorder.

The term “surfactant” as used herein refers to an amphiphilic compound or mixture of amphiphilic compounds that lowers the surface tension (or interfacial tension) between two liquids or between a liquid and a solid.

The term “and/or” as used herein refers to and encompasses any and all possible combinations of one or more of the associated listed items.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “SNEDD” or “self-nanoemulsifying drug delivery” as used herein refers to compositions which upon contact with aqueous media forms a nanoemulsion, on its own or with mild agitation.

The term “nanoemulsion” or “nanoemulsifying” as used herein refers to an emulsion having an average droplet size (mean diameter) less than about 1000 nm.

The term “emulsion” or “emulsifying” as used herein refers to a colloidal dispersion of two immiscible liquids, for example, an oil and water (or other aqueous liquid), or to the formation thereof.

The term “cannabinoid” as used herein refers to one of a group of compounds that acts on cannabinoid receptors.

The term “cannabinoid extract” as used herein refers to an extract from a cannabis plant comprising one or more cannabinoids.

The term “hydrophilic-lipophilic balance (HLB)” as used herein refers to a measure of the degree to which a surfactant is lipophilic or hydrophilic.

II. Formulations and Compositions of the Application

Emulsifying drug-delivery formulations, for example, self-emulsifying drug delivery formulations comprising a cannabinoid, a mixture of cannabinoids or a cannabinoid extract have been prepared. In an embodiment, liquid and solid emulsifying drug-delivery formulations have been prepared.

In an embodiment, the Applicants have found that the liquid emulsifying drug-delivery formulations can be converted into a solid emulsifying formulations that are, for example, free flowing powders, by, for example, plating, spray drying, hot-melt extrusion or spray congealing the liquid emulsifying formulation onto the one or more water soluble carrier solid materials. In an embodiment, the Applicants have surprisingly found that the liquid emulsifying drug-delivery formulations can be converted into a solid emulsifying formulations that are, for example, free flowing powders, by plating the liquid emulsifying formulation onto one or more water soluble carrier solid materials such as sorbitol. In some embodiments, when the one or more water soluble carrier solid materials is sorbitol, the plating is performed in the presence of water. Plating the liquid emulsifying formulations onto sorbitol in the presence of water have been to produce solid emulsifying formulations as dry powders comprising the liquid emulsifying formulation adsorbed onto the surface and/or coated inside of the pores of the solid support in dry form, and not wet powders of soluble carrier solid material coated with the liquid emulsifying formulation.

In an embodiment, the liquid and solid emulsifying drug-delivery formulations drug-delivery formulations of the application are non-aqueous emulsifying drug-delivery formulations which have been found to be able to spontaneously disperse in aqueous media at room temperature to form emulsions, for example, nanoemulsions comprising submicron sized droplets, without agitation or upon mild agitation. The emulsion produced by the emulsifying formulations of present application was found to increase the solubility of the hydrophobic cannabinoid, mixture of cannabinoids and/or cannabinoid extract, thereby enhancing the oral bioavailability of the active(s). Further, the emulsifying formulations of the application were found to have enhanced stability, clarity and fast on-set of action.

The emulsifying formulations have of the application are further compatible with a large variety of food and beverage products. Accordingly, the emulsifying formulation can be used as food and beverage product additives. In an embodiment, the solid emulsifying formulation is in a powder form which is easy to transport and therefore more convenient for a consumer. In an embodiment, the emulsifying formulations of the application can be mixed with an aqueous medium prior to administration or use.

Accordingly, the present application includes a liquid emulsifying formulation comprising:
i) one or more cannabinoids, and/or a cannabinoid extract; and
ii) one or more surfactants;
wherein the weight ratio of the one or more surfactants to the one or more cannabinoids, and/or the cannabinoid extract is from about 1:5 to about 100:1.

In an embodiment, the emulsifying formulation optionally further comprises one or more oils. In an embodiment, the weight ratio of the one or more oils to the one or more cannabinoids, and/or the cannabinoid extract is from about 1:99.9 to about 99.9:1. Accordingly, the present application also includes a liquid emulsifying formulation comprising:
i) one or more cannabinoids, and/or a cannabinoid extract;
ii) one or more surfactants; and
iii) one or more oils,
wherein the weight ratio of the one or more surfactants to the one or more cannabinoids, and/or the cannabinoid extract is from about 1:5 to about 100:1, and the weight ratio of the one or more oils to the one or more cannabinoids, and/or the cannabinoid extract is from about 1:99.9 to about 99.9:1.

In an embodiment, the present application includes a liquid emulsifying formulation comprising:
i) one or more cannabinoids, and/or a cannabinoid extract;
ii) one or more surfactants selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof; and
iii) one or more oils selected from medium chain triglycerides (MCTs) oils and long chain triglycerides (LCTs) oils, and combinations thereof;
wherein the weight ratio of the one or more surfactants to the one or more cannabinoids, and/or the cannabinoid extract is from about 1:5 to about 100:1; and the weight ratio of the one or more oils to the one or more cannabinoids, and/or the cannabinoid extract is from about 1:99.9 to about 99.9:1.

In an embodiment, the emulsifying formulation further optionally comprises one or more co-surfactants. In an embodiment, the weight ratio of the one or more co-surfactants to the one or more surfactants is from about 1:99.9 to about 99.9:1. Accordingly, the present application also includes a liquid emulsifying formulation comprising:
i) one or more cannabinoids, and/or a cannabinoid extract;
ii) one or more surfactants;
iii) optionally, one or more cosurfactants; and
iv) one or more oils,
wherein the weight ratio of the one or more surfactants to the one or more cannabinoids, and/or the cannabinoid extract is from about 1:5 to about 100:1; the weight ratio of the one or more oils to the one or more cannabinoids, and/or the cannabinoid extract is from about 1:99.9 to about 99.9:1; and the weight ratio of the one or more co-surfactants to the one or more surfactants is from about 1:99.9 to about 99.9:1.

In an embodiment, one or more cannabinoids, and/or a cannabinoid extract is substantially solubilized by one or more surfactants, one or more cosurfactants; and/or the one or more oils.

In an embodiment, the liquid emulsifying formulation is a viscous liquid emulsifying formulation at room temperature. In an embodiment, the liquid emulsifying formulation is a semi-solid emulsifying formulation at room temperature.

By “room temperature”, it is meant, for example, about 18 degrees celcius to about 25 degrees celcius. By “semi-solid”, it is meant, for example, that the emulsifying formulation is non-flowable and may be deformed when acted upon by force.

The Applicants have shown that a liquid emulsifying formulation can be converted into a dry solid emulsifying formulation by, for example, plating, spray drying, hot-melt extrusion or spray congealing the liquid emulsifying formulation onto one or more water soluble carrier solid materials. In an embodiment, the liquid self-emulsifying formulation is adsorbed onto one or more water soluble carrier materials to produce a solid self-emulsifying formulation. In an embodiment, the solid emulsifying formulations produced by, for example, plating comprises the liquid emulsifying formulation adsorbed onto the surface and/or coated inside of the pores of the solid support in dry form. Therefore, in an embodiment, the formulation further optionally comprises one or more water soluble carrier solid materials.

Accordingly, the present application includes a solid emulsifying formulation comprising:
i) one or more cannabinoids, and/or a cannabinoid extract;
ii) one or more surfactants; and
iii) one or more water soluble carrier solid materials,
wherein the weight ratio of the one or more surfactants to the one or more cannabinoids, and/or the cannabinoid extract is from about 1:5 to about 100:1.

In an embodiment, the solid emulsifying formulation optionally further comprises one or more oils. In an embodiment, the weight ratio of the one or more oils to the one or more cannabinoids, and/or the cannabinoid extract is from about 1:99.9 to about 99.9:1. Accordingly, the present application also includes a solid emulsifying formulation comprising:
i) one or more cannabinoids, and/or a cannabinoid extract;
ii) one or more surfactants;
iii) one or more oils, and
iv) one or more water soluble carrier solid materials,
wherein the weight ratio of the one or more surfactants to the one or more cannabinoids, and/or the cannabinoid extract is from about 1:5 to about 100:1, and the weight ratio of the one or more oils to the one or more cannabinoids, and/or the cannabinoid extract is from about 1:99.9 to about 99.9:1.

In an embodiment, the present application includes a solid emulsifying formulation comprising:
i) one or more cannabinoids, and/or a cannabinoid extract;
ii) one or more surfactants selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof;
iii) one or more oils selected from medium chain triglyceride (MCT) oils and long chain triglycerides (LCT) oils, and combinations thereof; and
iv) one or more water soluble carrier solid materials selected from one or more sugars, sugar alcohols, oligosaccharides, and polysaccharides, and combinations thereof,
wherein the weight ratio of the one or more surfactants to the one or more cannabinoids, and/or the cannabinoid extract is from about 1:5 to about 100:1; and the weight ratio of the one or more oils to the one or more cannabinoids, and/or the cannabinoid extract is from about 1:99.9 to about 99.9:1.

In an embodiment, the solid emulsifying formulation comprises one or more co-surfactants. In an embodiment, the weight ratio of the one or more co-surfactants to the one or more surfactants is from about 1:99.9 to about 99.9:1.

Accordingly, the present application also includes a solid emulsifying formulation comprising:
i) one or more cannabinoids, and/or a cannabinoid extract;
ii) one or more surfactants;
iii) optionally, one or more cosurfactants;
iv) one or more oils, and
v) one or more water soluble carrier solid materials,
wherein the weight ratio of the one or more surfactants to the one or more cannabinoids, and/or the cannabinoid extract is from about 1:5 to about 100:1; the weight ratio of the one or more oils to the one or more cannabinoids, and/or the cannabinoid extract is from about 1:99.9 to about 99.9:1; and the weight ratio of the one or more co-surfactants to the one or more surfactants is from about 1:99.9 to about 99.9:1.

In an embodiment, the present application includes a solid emulsifying formulation comprising:
i) one or more cannabinoids, and/or a cannabinoid extract;
ii) one or more surfactants selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof;
iii) one or more cosurfactants,
iv) one or more oils selected from medium chain triglycerides (MCT) oils and long chain triglycerides (LCT) oils, and combinations thereof; and
v) one or more water soluble carrier solid materials selected from one or more sugars, sugar alcohols, oligosaccharides, and polysaccharides, and combinations thereof,
wherein the weight ratio of the one or more surfactants to the one or more cannabinoids, and/or the cannabinoid extract is from about 1:5 to about 100:1; the weight ratio of the one or more oils to the one or more cannabinoids, and/or the cannabinoid extract is from about 1:99.9 to about 99.9:1, and the weight ratio of the one or more co-surfactants to the one or more surfactants is from about 1:99.9 to about 99.9:1.

In an embodiment, the solid emulsifying formulation is a powder. In an embodiment, the solid emulsifying formulation is a free-flowing powder. Accordingly, the present application also include a powder or free-flowing solid emulsifying formulation as described above.

It would be appreciated by a person skilled in the art that the solid emulsifying formulations, for example, powder or free-flowing powder emulsifying formulations are easier to transport and formulate in comparison to the liquid emulsifying formulations.

In an embodiment, the emulsifying formulations of the application are self-emulsifying formulations. In an embodiment, the emulsifying formulations of the application are self-microemulsifying formulations. In an embodiment, the emulsifying formulations of the application are self-nanoemulsifying formulations.

In an embodiment, the liquid and solid emulsifying formulations of the application are non-aqueous formulations. In an embodiment, the liquid and solid emulsifying formulations of the application are essentially or completely free of water, and are mixed with an aqueous medium prior to administration or use. In an embodiment, the solid emulsifying formulations of the application comprises the liquid emulsifying formulation adsorbed onto the surface and/or coated inside of the pores of the solid support to form a powder such as a free flowing powder.

By “essentially free” of water, it is meant, for example, that the emulsifying formulation comprises less than about 5% water.

In an embodiment, the liquid and solid emulsifying formulations of the application self-emulsify upon contact with an aqueous medium to produce an emulsion. In an embodiment, the emulsion comprises a plurality of droplets. In an embodiment, the liquid and solid emulsifying formulations of the application self-emulsify upon contact with an aqueous medium to produce a submicron emulsion comprising a plurality of droplets. In an embodiment, liquid and solid emulsifying formulations of the application self-emulsify spontaneously or under mild agitation (such as shaking, mixing or stirring).

In an embodiment, the liquid and solid emulsifying formulations of the application self-emulsify in water at 25 degree celcius within less than 30 minutes, less than 20 minutes, less than 15 minutes, less than 10 minutes, less than 5 minutes, less than 3 minutes, less than 2 minutes, less than 1 minute, less than 45 seconds, less than 30 seconds, less than 15 seconds, or less than 10 seconds on its own or with mild agitation. In an embodiment, the liquid and solid emulsifying formulations of the application self-emulsify in water at 25 degree celcius within less than 1 minute, less than 45 seconds, less than 30 seconds, or less than 15 seconds, or less than 10 seconds on its own or with mild agitation. In an embodiment, the emulsifying formulations of the application self-emulsify in water at 25 degree celcius within less than 30 seconds on its own or with mild agitation.

In an embodiment, at least 80% of the liquid and solid emulsifying formulations of the application self-emulsify in water at 25 degree celcius within less than 15 minutes, less than 10 minutes, less than 5 minutes, less than 3 minutes, less than 2 minutes, less than 1 minutes, less than 45 seconds, less than 30 seconds, less than 15 seconds, or less than 10 seconds. In an embodiment, the liquid and solid emulsifying formulations of the application self-emulsify in water at 25 degree celcius within less than 1 minute, less than 45 seconds, less than 30 seconds, or less than 15 seconds, or less than 10 seconds. In an embodiment, the emulsifying formulations of the application self-emulsify in water at 25 degree celcius within less than 30 seconds.

In an embodiment, the liquid and solid emulsifying formulations of the application self-emulsify upon contact with an aqueous medium to produce a stable emulsion. In an embodiment, the liquid and solid emulsion comprises a plurality of droplets. In an embodiment, the droplets have an average particle size (mean diameter) of about 10 nm to about 500 nm. In an embodiment, the droplets have an average particle size of about 10 nm to about 150 nm. In an embodiment, the droplets have an average particle size of about 10 nm to about 100 nm. In an embodiment, the droplets have an average particle size of about 20 nm to about 100 nm. In an embodiment, the droplets have an average particle size of about 20 nm to about 80 nm. In an embodiment, the droplets have an average particle size of about 20 nm to about 60 nm. In an embodiment, the liquid and solid emulsifying formulations of the application emulsifies in an aqueous medium to produce an emulsion that is clear and/or transparent.

In an embodiment, the liquid and solid emulsifying formulations of the application self-emulsify upon contact with an aqueous medium to produce an emulsion that is stable are after centrifugation for at least 60 minutes at 6000 rpm.

In an embodiment, the weight ratio of the one or more surfactants to the one or more cannabinoids, and/or the cannabinoid extract is from about 1:3 to about 75:1. In an embodiment, the weight ratio of the one or more surfactants to the one or more cannabinoids and/or the cannabinoid extract and the one or more oils is from about 1:1 to about 75:1. In an embodiment, the weight ratio of the one or more surfactants to the one or more cannabinoids and/or the cannabinoid extract and the one or more oils is from about 1:1 to about 50:1, about 1:1 to about 40:1, about 1:1 to about 30:1, about 1:1 to about 20:1, about 1:1 to about 15:1, about 5:1 to about 50:1, about 5:1 to about 40:1, about 5:1 to about 30:1, about 5:1 to about 25:1, about 5:1 to about 20:1, about 5:1 to about 15:1, about 10:1 to about 50:1, about 10:1 to about 40:1, about 10:1 to about 30:1, about 10:1 to about 20:1, about 10:1 to about 15:1, about 10:1 to about 13:1, or about 12:1 In an embodiment, the weight ratio of the one or more surfactants to the one or more cannabinoids, and/or the cannabinoid extract is from about 3:1 to about 50:1. In an embodiment, the weight ratio of the one or more surfactants to the one or more cannabinoids, and/or the cannabinoid extract is from about 3:1 to about 20:1. In an embodiment, the weight ratio of the one or more surfactants to the one or more cannabinoids and/or the cannabinoid extract and the one or more oils is from about 5:1 to about 20:1, about 5:1 to about 15:1, about 10:1 to about 20:1, about 10:1 to about 15:1, about 10:1 to about 13:1, or about 12:1.

In an embodiment, the weight ratio of the one or more cannabinoids, and/or the cannabinoid extract, the one or more carrier oils and the one or more surfactants to the one or more solid carriers is from about 1:1 to about 1:1000. In an embodiment, the weight ratio is from about 1:1 to about 1:100. In an embodiment, the weight ratio of the one or more cannabinoids, and/or the cannabinoid extract, the one or more carrier oils and the one or more surfactants to the one or more solid carriers is from about 1:1 to about 1:50. In an embodiment, the weight ratio of the one or more cannabinoids, and/or the cannabinoid extract, the one or more carrier oils and the one or more surfactants to the one or more solid carriers is from about 1:2 to about 1:40.

A person of skill in the art would appreciate the weight ratio of one or more cannabinoids, and/or a cannabinoid extract, the one or more oils and the one or more surfactants to the one or more solid carriers would depend on the surface area of the solid carrier and the desired flowability of the formulation.

In an embodiment, the weight ratio of the one or more oils to the one or more cannabinoids and/or the cannabinoid extract is from about 1:75 to about 75:1. In an embodiment, the weight ratio is from about 1:50 to about 50:1. In an embodiment, the weight ratio of the one or more oils to the one or more cannabinoids and/or the cannabinoid extract is from about 1:10 to about 10:1. In an embodiment, the weight ratio is from about 1:1 to about 10:1. In an embodiment, the weight ratio of the one or more oils to the one or more cannabinoids and/or the cannabinoid extract is from about 1:1 to about 4:1. In an embodiment, the weight ratio of the one or more oils to the one or more cannabinoids and/or the cannabinoid extract is from about 2:1 to about 3:1. In an embodiment, the weight ratio of the one or more oils to the one or more cannabinoids and/or the cannabinoid extract is from about 2:1 to about 3:1. In an embodiment, the weight ratio of the one or more oils to the one or more cannabinoids and/or the cannabinoid extract is from about 3:1.

In an embodiment, the weight ratio of the one or more surfactants to the one or more cannabinoids and/or the cannabinoid extract and the one or more oils is from about 100: to about 1:100. In an embodiment, the weight ratio of the one or more surfactants to the one or more cannabinoids and/or the cannabinoid extract and the one or more oils is from about 50:1 to about 1:50. In an embodiment, the weight ratio of the one or more surfactants to the one or more cannabinoids and/or the cannabinoid extract and the one or more oils is from about 10:1 to about 1:10. In an embodiment, the weight ratio of the one or more surfactants to the one or more cannabinoids and/or the cannabinoid extract and the one or more oils is from about 5:1 to about 1:5. In an embodiment, the weight ratio of the one or more surfactants to the one or more cannabinoids and/or the cannabinoid extract and the one or more oils is from about 1:1 to about 1:5.

In an embodiment, the weight ratio of the one or more surfactants to the one or more oils is from about 100: to about 1:100. In an embodiment, the weight ratio of the one or more surfactants to the one or more oils is from about 50:1 to about 1:50. In an embodiment, the weight ratio of the one or more surfactants to the one or more oils is from about 10:1 to about 1:10. In an embodiment, the weight ratio i of the one or more surfactants to the one or more oils is from about 10:1 to about 1:1. In an embodiment, the weight ratio of the one or more surfactants to the one or more oils is from about 7:1 to about 1:1.

In an embodiment, the weight ratio of the one or more co-surfactants to the one or more surfactants is from about 1:20 to about 5:1. In an embodiment, the weight ratio of the one or more co-surfactants to the one or more surfactants is from about 1:10 to about 1:1. In an embodiment, if present, the weight ratio of one or more co-surfactants to the one or more surfactants is from about 1:10 to about 1:5.

In an embodiment, the weight ratio of the one or more oils to the one or more cannabinoids, and/or the cannabinoid extract is from about 1:10 to about 10:1, the weight ratio of the one or more surfactants to the one or more cannabinoids, and/or the cannabinoid extract is from about 1:3 to about 75:1 and if present, the weight ratio of the one or more co-surfactants to the one or more surfactants is from about 1:20 to about 5:1.

In an embodiment, the weight ratio of the one or more oils to the one or more cannabinoids, and/or the cannabinoid extract is from about 1:1 to about 10:1, the weight ratio of the one or more surfactants to the one or more cannabinoids, and/or the cannabinoid extract is from about 3:1 to about 50:1 and if present, the weight ratio of the one or more co-surfactants to the one or more surfactants is from about 1:10 to about 1:1:

In an embodiment, the weight ratio of the one or more oils to the one or more cannabinoids, and/or the cannabinoid extract is from about 1:1 to about 4:1, the weight ratio of the one or more surfactants to the one or more cannabinoids, and/or the cannabinoid extract is from about 3:1 to about 20:1 and if present, the weight ratio of the one or more co-surfactants to the one or more surfactants is from about 1:10 to about 1:5.

In an embodiment, the weight ratio of the one or more oils to the one or more cannabinoids, and/or the cannabinoid extract from about 2:1 to about 3:1, the weight ratio of the one or more surfactants to the one or more cannabinoids, and/or the cannabinoid extract is from about 10:1 to about 15:1, and the weight ratio of the one or more co-surfactants to the one or more surfactants is from about 1:10 to about 1:5.

In an embodiment, the cannabinoid extract is obtained from a cannabis plant selected from Cannabis sativa, Cannabis indica, and Cannabis hybrid. In an embodiment, the cannabinoid extract is an extract of Cannabis sativa. In an embodiment, the cannabinoid extract is obtained from a cannabis plant by supercritical CO₂ extraction, subcritical CO₂ extraction, or organic solvent extraction.

In an embodiment, the cannabinoid extract is obtained from a cannabis plant without solvent such as a mechanic press, a hydraulic separator, or a sieve. In an embodiment, the cannabinoid extract is a dry sift.

In an embodiment, the cannabinoid extract obtained from a cannabis plant is purified. In an embodiment, the cannabinoid extract is purified by dewaxing. In an embodiment, the cannabinoid extract is purified by fractional distillation. In an embodiment, the cannabinoid extract is a cannabinoid distillate. In an embodiment, the cannabinoid distillate is the product of short path distillation.

In an embodiment, the cannabinoid extract is purified such that one or more cannabinoid is present at greater than 95% of the total extract (w/w). In an embodiment, the cannabinoid extract is purified such that one or more cannabinoid is present at greater than 98% of the total extract (w/w).

In an embodiment, the one or more cannabinoids are synthetic.

In an embodiment, the one or more cannabinoids are selected from one of more of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBT), tetrahydrocannabinol (THC), tetrahydrocannabinolic acid (THCA), tetrahydrocannabivarin (THCV) and tetrahydrocannabivarinic acid (THCVA), and combinations thereof.

In an embodiment, the one or more cannabinoids are selected from one of more of CBD and THC, and combinations thereof. In an embodiment, the one or more cannabinoids is CBD. In an embodiment, the one or more cannabinoids is THC. In an embodiment, the one or more cannabinoids is a combination of CBD and THC. In an embodiment, the CBD and THC are in a ratio of from about 1:100 to about 100:1, about 1:10 to about 10:1, about 1:3 to about 3:1, about 1:2 to about 2:1 or about 1:1.

In an embodiment, the one or more cannabinoids, and/or a cannabinoid extract is selected from CBD, THC, and a cannabinoid distillate and combinations thereof. In an embodiment, the one or more cannabinoids, and/or a cannabinoid extract is CBD. In an embodiment, the one or more cannabinoids, and/or a cannabinoid extract is THC. In an embodiment, the one or more cannabinoids, and/or a cannabinoid extract is a combination of CDB and THC. In an embodiment, the one or more cannabinoids, and/or a cannabinoid extract is a cannabinoid distillate

In an embodiment, the one or more oils are selected from one or more oils comprising fatty acids, monoglycerides, diglycerides, triglycerides or combinations thereof. In an embodiment, the one or more oils are oils comprising triglycerides, hydrolyzed triglycerides, propylene glycol mono esters, or propylene glycol diesters, or combinations thereof. In an embodiment, the hydrolyzed triglyceride is a monoglyceride or a diglyceride, or combinations thereof. In an embodiment, the one or more oils are water-insoluble oils including, but not limited to, naturally occurring terpenes or essential oils of natural sources. In an embodiment, the one or oils are benzyl alcohol, 1,3-butylene, citric acid esters of mono- and di-glycerides, ethyl acetate, glyceryl diacetate, glyceryl triacetate, glyceryl tributyrate, triethyl, or combinations thereof.

In an embodiment, the oils comprising triglycerides are oils comprising long chain triglycerides (LCT) and/or medium chain triglycerides (MCT). In an embodiment, the one or more oils are oils comprising long chain triglycerides (LCT oils) or medium chain triglycerides (MCT oils) or combinations thereof.

In an embodiment, LCTs are triglycerides whose fatty acids have an aliphatic tail of 13-24 carbon atoms. In an embodiment, the LCTs are formed from long chain fatty having from C14 to C16, C16 to C18, C18 to C20, C14 to C20, or C20 to C24 atoms. In an embodiment, the fatty acids of the LCTs may be saturated, mono-unsaturated, and poly-unsaturated fatty acids. In one embodiment, 5 to 25% of the long chain fatty acids are saturated, 15 to 80% are monounsaturated, and 15 to 80% are polyunsaturated. In an embodiment, the oils comprising an LCT (LCT oil) may comprise at least 5 wt % long chain triglycerides. In an embodiment, the LCT oils are selected from olive oil, poppy seed, safflower, sunflower, corn, soybean, sesame oil, or castor oil, or combinations thereof. In an embodiment, LCTs oils may be in the form of oil that is enriched or fractionated to increase the concentration of long chain triglycerides. In an embodiment, the LCT oil is selected from soybean oil, sunflower oil, olive oil, palm oil, coconut oil, canola oil, peanut oil, cottonseed oil, corn oil, safflower oil, sesame oil, grape seed oil, hemp seed oil, and flaxseed oil, and combinations thereof. In an embodiment, the LCT oil is sunflower oil.

In an embodiment, the MCTs are triglycerides whose fatty acids have an aliphatic tail of 6-12 carbon atoms. In an embodiment, the MCTs are formed from fatty acids having from C6 to C8, C8 to C10, C10 to C12, or C8 to C12 carbon atoms. In an embodiment, the MCT may be saturated, mono-unsaturated, and/or poly-unsaturated fatty acids. In an embodiment, 80% to 100% of the medium chain fatty acids are saturated, 0 to 10% are monounsaturated, and 0 to 5% are polyunsaturated. In an embodiment, medium chain fatty acids include caproic acid, caprylic acid, capric acid, and mixtures thereof. In an embodiment, an oil comprising MCT (MCT oil), may comprise at least 5 wt % medium chain triglycerides. In an embodiment, an oil comprising MCT (MCT oil) is coconut oil, or palm kernel oil, or combinations thereof. In an embodiment, the oil comprising an MCT (MCT oil) is coconut oil. In an embodiment, the MCT oil may be in the form of oil that is enriched or fractionated to increase the concentration of medium chain triglycerides. In an embodiment, the MCT oil is fractionated coconut oil. In an embodiment, the fractionated coconut oil is glyceryl tricaprylate. In an embodiment, the medium chain triglycerides may also be formed by esterifying glycerol with mixtures of C6-C12 fatty acids, for example, C8-C10 fatty acids such as caprylic (C:8) and capric (C:10) fatty acids fractionated from coconut or palm kernel oils. In an embodiment, an oil comprising MCT (MCT oil) is coconut oil, palm kernel oil, enriched form of coconut oil or palm kernel oil, and a fractionated form of coconut oil or palm kernel or combinations thereof. In an embodiment, the oil comprising an MCT (MCT oil) is coconut oil or fractionated form thereof. In an embodiment, the oil comprising an MCT (MCT oil) is fractionated coconut oil.

Accordingly, in an embodiment, the one or more oils are selected from medium chain triglyceride (MCT) oils and long chain triglyceride (LCT) oils, and combinations thereof. In an embodiment, the one or more oils are a combination of a MCT oil and a LCT oil. In an embodiment, the one or more oils are LCT. In an embodiment, the one or more oils is a MCT oil.

In an embodiment, an oil comprising MCT (MCT oil) is selected from coconut oil, palm kernel oil, enriched form of coconut oil or palm kernel oil, a fractionated form of coconut oil or palm kernel and combinations thereof. In an embodiment, the oil comprising an MCT (MCT oil) is coconut oil or a fractionated form thereof. In an embodiment, the oil comprising an MCT (MCT oil) is fractionated coconut oil. In an embodiment, the LCT oil is selected from soybean oil, sunflower oil, olive oil, palm oil, coconut oil, canola oil, peanut oil, cottonseed oil, corn oil, safflower oil, sesame oil, grape seed oil, hemp seed oil, and flaxseed oil, and combinations thereof. In an embodiment, the one or more oils are a combination of fractionated coconut oil and sunflower oil.

In an embodiment, the solid and liquid emulsifying formulation of the application emulsifies in an aqueous medium to produce an emulsion having a smaller average particle size compared to an otherwise identical emulsifying formulation of the application except without the one or more oils.

In an embodiment, the emulsifying formulation of the application comprises one or two oils. In an embodiment, the emulsifying formulation of the application comprises one oil. In an embodiment, the one or more oils are liquid at room temperature or the one or more oils are liquefied during the manufacturing process. In an embodiment, the one or more oils are a liquid at 25° C. In an embodiment, the one or more or more oil are liquefied when heated up to about 40° C. to about 60° C.

It would be appreciated by a person skilled in the art that the one or more oils is an exogenously added oil, and is other than any oil that may be naturally present in the one or more cannabinoids and/or cannabinoid extract.

In an embodiment, the one or more surfactants are monoglycerides, diglycerides, polysorbates, acetylated tartaric acid esters of monoglycerides, acetylated tartaric acid esters of diglycerides, citric acid esters of monoglycerides, citric acid esters of diglycerides, lecithins, hydroxylated lecithins, hydrolyzed lecithins, hydroxypropyl cellulose, lactylated monoglycerides, lactylated diglycerides, lactylic esters of fatty acids, polyglycerol esters of fatty acids or polyglycerol fatty acid esters (PGFE), polyglycerol esters of interesterified castor oil fatty acids, polyoxyethylene fatty esters, propylene glycol alginates, propylene glycol ether of methylcellulose, propylene glycol mono fatty acid esters, quillaia extract, sodium stearoyl-2-lactylate, sodium stearate, steelyl monoglyceridyl citrate, D-α-Tocopherol polyethylene glycol 1000 succinate (Vitamin E TPGS), polyoxyl hydrogenated castor oils, alkyl polyglycosides or sucrose esters of fatty acids or combinations thereof.

In an embodiment, the one or more surfactants are selected from: PEG 15 hydroxystearate, polyoxyl-10-Oleyl Ether, polyethylene glycol, hydrogenated castor oil, polyethylene glycol (PEG) hydrogenated castor oil, polyethylene-polypropylene glycol, PEG 8 caprylic/capric glycerides, PEG 300 oleic glycerides, diethylene glycol monoethyl ether, lauroyl macrogol 32 glycerides, polyethylene glycol 400 (PEG 400), propylene glycol laurate, D-α-Tocopherol polyethylene glycol 1000 succinate, polyethylene-polypropylene glycol, polyethylene-polypropylene glycol, polyvinyl pyrrolidone, Iota Carrageenan, Xanthan gum, locust Bean gum, Kelcogel LT100, acacia gum, guar gum, gamma-Cyclodextrin, Tracacanth gum, hydroxypropyl methylcellulose, carboxymethyl cellulose, microcrystalline cellulose, lecithin, polyethylene-polypropylene glycol, sucrose laurate, sucrose palmitate, sucrose stearate, gamma-cyclodextrin, beta-cyclodextrin, pectin, whey protein, caseinates, quillaia/quillaja saponins, quillaia extract, PEG 8 stearate, and PEG 40 stearate, and combinations thereof.

In an embodiment, the one or more surfactants are polysorbates. In an embodiment, the polysorbates are polyethylene glycol sorbitan monolaurate (polysorbate 20), polyethylene glycol sorbitan mono palmitate (polysorbate 40), polyethylene glycol sorbitan monostearate (polysorbate 60), polyethylene glycol sorbitan tristearate (polysorbate 65), polyethylene glycol sorbitan monooleate (polysorbate 80), polyethylene glycol sorbitan trioleate (polysorbate 85) polyethylene glycol sorbitan hexaoleate, polyethylene glycol sorbitan tetraoleate, sorbitan monolaurate (Span 20), sorbitan monopalmitate (Span 40), sorbitan monostearate (Span 60), sorbitan tristearate (Span 65), sorbitane monooleate (Span 80), or sorbitan trioleate (Span 85), or combinations thereof. In an embodiment, the polysorbates are polyethylene glycol sorbitan monolaurate (polysorbate 20), polyethylene glycol sorbitan mono palmitate (polysorbate 40), polyethylene glycol sorbitan monostearate (polysorbate 60), polyethylene glycol sorbitan tristearate (polysorbate 65), polyethylene glycol sorbitan monooleate (polysorbate 80), polyethylene glycol sorbitan trioleate (polysorbate 85) polyethylene glycol sorbitan hexaoleate or polyethylene glycol sorbitan tetraoleate, or combinations thereof. In an embodiment, the polysorbates are polyethylene glycol sorbitan monolaurate (polysorbate 20), polyethylene glycol sorbitan mono palmitate (polysorbate 40), polyethylene glycol sorbitan monostearate (polysorbate 60), polyethylene glycol sorbitan tristearate (polysorbate 65), polyethylene glycol sorbitan monooleate (polysorbate 80), polyethylene glycol sorbitan trioleate (polysorbate 85), or combinations thereof. In an embodiment, the polysorbate is polyethylene glycol sorbitan monooleate (polysorbate 80).

In an embodiment, the one or more surfactants are polyethylene glycol (PEG) hydrogenated castor oils, also known as polyoxyethylene hydrogenated castor oils. In an embodiment, the polyoxyethylene hydrogenated castor oils are polyoxyl (20) hydrogenated castor oil, polyoxyl (25) hydrogenated castor oil, polyoxyl (30) hydrogenated castor oil, polyoxyl (35) hydrogenated castor oil, polyoxyl (40) hydrogenated castor oil, polyoxyl (45) hydrogenated castor oil, polyoxyl (50) hydrogenated castor oil, polyoxyl (55) hydrogenated castor oil or polyoxyl (60) hydrogenated castor oil, or combinations thereof. In an embodiment, the polyoxyethylene hydrogenated castor oils are polyoxyl (20) hydrogenated castor oil, polyoxyl (25) hydrogenated castor oil, polyoxyl (30) hydrogenated castor oil, polyoxyl (35) hydrogenated castor oil, polyoxyl (40) hydrogenated castor oil, polyoxyl (45) hydrogenated castor oil, polyoxyl (50) hydrogenated castor oil, polyoxyl (55) hydrogenated castor oil or polyoxyl (60) hydrogenated castor oil, or combinations thereof. In an embodiment, the polyoxyethylene hydrogenated castor oils are polyoxyl (35) hydrogenated castor oil, polyoxyl (40) hydrogenated castor oil or polyoxyl (60) hydrogenated castor oil, or combinations thereof. In an embodiment, the polyoxyethylene hydrogenated castor oil is polyoxyl (40) hydrogenated castor oil.

In an embodiment, the one or more surfactants is selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof. In an embodiment, the one or more surfactants is selected from polysorbates, polyoxyethylene hydrogenated castor oils, and D-α-Tocopherol polyethylene glycol 1000 succinate (Vitamin E TPGS), and combinations thereof. In an embodiment, the polysorbate is polyethylene glycol sorbitan monooleate (polysorbate 80). In an embodiment, the lecithin is hydrolyzed lecithins.

In an embodiment, the one or more surfactants have a hydrophilic-Lipophilic Balance (HLB) that is greater than 10. In an embodiment, the one or more surfactants have a hydrophilic-lipophilic balance (HLB) that is greater than 12.

In an embodiment, the one or more surfactants are natural surfactants. In an embodiment, the one or more surfactants are artificial surfactants. In an embodiment, the one or more surfactants are water soluble.

In an embodiment, the emulsifying formulation of the application comprises one surfactant.

In embodiment, the one or more co-surfactants are medium chain alcohols, medium chain organic acids, propylene glycol, ethanol, propanol, butanol, pentanol, hexanol, glycerin, benzyl alcohol, isopropanol, phenethyl alcohol, butylene glycol, polyethylene glycol 400 (PEG400), diethylene glycol monoethyl ether, poly(ethylene glycol) tetrahydrofurfuryl ether, N-methyl pyrrolidone, sodium deoxycholate, caprylic acid, sodium caprylate, or potassium sorbate, or combinations thereof. In embodiment, the co-surfactant is propylene glycol, ethanol, propanol, butanol or glycerol, or combinations thereof. In embodiment, the co-surfactant is propylene glycol.

In an embodiment, the one or more co-surfactants are water soluble. In an embodiment, the water soluble co-surfactants act as water soluble solid carriers.

In an embodiment, the one or more co-surfactants increases the dispersibility of the emulsifying formulation compared to an otherwise identical emulsifying formulation of the application except without the one or more co-surfactants.

In an embodiment, the one or more water soluble solid carrier materials are selected from one or more sugars, sugar alcohols, oligosaccharides and polysaccharides, or combinations thereof. In an embodiment, the one or more water soluble solid carrier materials are selected from one or more sugars, sugar alcohols, and polysaccharides, or combinations thereof. In an embodiment, the one or more water soluble solid carrier materials are selected from one or more sugars and sugar alcohols, and combinations thereof. In an embodiment, the one or more water soluble solid carrier materials are one or more sugar alcohols.

In an embodiment, the one or more sugars are any monosaccharides or disaccharides. In an embodiment, the one or more sugars are selected from sucrose, glucose, fructose, galactose, maltose, lactose, mannose, ribose, rhamnose, trehalose, and xylose, and combinations thereof. In an embodiment, the one or more sugars are selected from sucrose, glucose, and fructose, and combinations thereof.

In an embodiment, the one or more “sugar alcohol” is any sugar whose reducing terminal is reduced. In an embodiment, the one or more sugar alcohol is selected from sorbitol, lactitol, maltitol, xylitol, mannitol, erythritol, ribitol, galactitol, fucitol, iditol, inositol, and lactitol, and combinations thereof. In an embodiment, the one or more sugar alcohols are selected from sorbitol, mannitol, xylitol, and erythritol, and combinations thereof. In an embodiment, the one or more sugar alcohols are selected from sorbitol, mannitol, and xylitol, and combinations thereof. In an embodiment, the sugar alcohol is sorbitol. In an embodiment, the sugar alcohol is mannitol.

In an embodiment, the one or more oligosaccharides are any saccharide comprising three to ten monosaccharide units. In an embodiment, the oligosaccharide is selected from fructo-oligosaccharides (FOS), galacto-oligosaccharides (GOS), gluco-oligosaccharides arabino-oligosaccharides, manno-oligosaccharides, xylo-oligosaccharides, isolmalto-oligosaccharides (IMO), raffinose family of oligosaccharides (RFO), gluco-galacto-oligosaccharides, gluco-fructo-oligosaccharides, gluco-manno-oligosaccharides, gluco-arabino-oligosaccharides, gluco-xylo-oligosaccharides, galacto-fructo-oligosaccharides, galacto-manno-oligosaccharides, galacto-arabino-oligosaccharides, galacto-xylo-oligosaccharides, fructo-manno-oligosaccharides, fructo-arabino-oligosaccharides, fructo-xylo-oligosaccharides, manno-arabino-oligosaccharides, manno-xylo-oligosaccharides, and arabino-xylo-oligosaccharides, and combinations thereof.

In an embodiment, the one or more polysaccharides are any saccharides comprising more than ten monosaccharide units. In an embodiment, the polysaccharide is selected from modified starches, maltodextrin, polydextrose, fructans, glucans, xylans and galactans and combinations thereof. In an embodiment, the polysaccharide is maltodextrin.

In an embodiment, the one or more water soluble solid carrier material has a melting point below 150° C.

Accordingly, in an embodiment, the present application includes a solid emulsifying formulation comprising:
i) one or more cannabinoids, and/or a cannabinoid extract;
ii) one or more surfactants selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof;
iii) one or more oils selected from medium chain triglyceride (MCT) oils and long chain triglycerides (LCT) oils, and combinations thereof;
iv) optionally, one or more cosurfactants; and
v) one or more water soluble carrier solid materials selected from one or more sugars, sugar alcohols, oligosaccharides, and polysaccharides, and combinations thereof,
wherein the weight ratio of the one or more oils to the one or more cannabinoids, and/or the cannabinoid extract is from about 1:1 to about 10:1, the weight ratio of the one or more surfactants to the one or more cannabinoids, and/or the cannabinoid extract is from about 3:1 to about 50:1 and if present, the weight ratio of the one or more co-surfactants to the one or more surfactants is from about 1:10 to about 1:1:

In an embodiment, the weight ratio of the one or more oils to the one or more cannabinoids, and/or the cannabinoid extract is from about 1:1 to about 4:1, the weight ratio of the one or more surfactants to the one or more cannabinoids, and/or the cannabinoid extract is from about 3:1 to about 20:1 and if present, the weight ratio of the one or more co-surfactants to the one or more surfactants is from about 1:10 to about 1:5

In an embodiment, the weight ratio of the one or more oils to the one or more cannabinoids, and/or the cannabinoid extract from about 2:1 to about 3:1, the weight ratio of the one or more surfactants to the one or more cannabinoids, and/or the cannabinoid extract is from about 10:1 to about 15:1, and the weight ratio of the one or more co-surfactants to the one or more surfactants is from about 1:10 to about 1:5.

In an embodiment, the one or more cannabinoids, and/or a cannabinoid extract is selected from CBD, THC, and a cannabinoid distillate and combinations thereof.

In an embodiment, the one or more oils are a combination of a MCT oil and a LCT oil. In an embodiment, the one or more oils is a MCT oil.

In an embodiment, an oil comprising MCT (MCT oil) is selected from coconut oil, palm kernel oil, enriched form of coconut oil or palm kernel oil, a fractionated form of coconut oil or palm kernel and combinations thereof. In an embodiment, the oil comprising an MCT (MCT oil) is coconut oil or a fractionated form thereof. In an embodiment, the oil comprising an MCT (MCT oil) is fractionated coconut oil.

In an embodiment, the LCT oil is selected from soybean oil, sunflower oil, olive oil, palm oil, coconut oil, canola oil, peanut oil, cottonseed oil, corn oil, safflower oil, sesame oil, grape seed oil, hemp seed oil, and flaxseed oil, and combinations thereof.

In an embodiment, the one or more oils are a combination of fractionated coconut oil and sunflower oil.

In an embodiment, the one or more surfactants is selected from polysorbates, polyoxyethylene hydrogenated castor oils, and D-α-Tocopherol polyethylene glycol 1000 succinate (Vitamin E TPGS), and combinations thereof.

In an embodiment, the polysorbate is polyethylene glycol sorbitan monooleate (polysorbate 80).

In embodiment, the co-surfactant is propylene glycol, ethanol, propanol, butanol or glycerol, or combinations thereof. In embodiment, the co-surfactant is propylene glycol.

In an embodiment, the one or more water soluble solid carrier materials are selected from one or more sugars, sugar alcohols, and polysaccharides, or combinations thereof. In an embodiment, the one or more water soluble solid carrier materials are selected from one or more sugars and sugar alcohols, and combinations thereof. In an embodiment, the polysaccharide is maltodextrin. In an embodiment, the one or more sugar alcohol is selected from sorbitol, lactitol, maltitol, xylitol, mannitol, erythritol, ribitol, galactitol, fucitol, iditol, inositol, and lactitol, and combinations thereof. In an embodiment, the one or more water soluble solid carrier materials are one ore more sugar alcohols. In an embodiment, the one or more sugar alcohols are selected from sorbitol and mannitol and combinations thereof. In an embodiment, the sugar alcohol is sorbitol.

In an embodiment, the one or more surfactants are selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof, the one or more oils are selected from medium chain triglyceride (MCT) oils and long chain triglycerides (LCT) oils, and combinations thereof, the one or more cosurfactants if present are selected from propylene glycol, ethanol, propanol, butanol and glycerol, and combinations thereof, and the one or more water soluble carrier solid materials are selected from one or more sugars, sugar alcohols, and polysaccharides, and combinations thereof. In an embodiment, the polysorbate is polysorbate 80.

In an embodiment, the one or more surfactants are selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof, the one or more oils are selected from medium chain triglyceride (MCT) oils and long chain triglycerides (LCT) oils, and combinations thereof, wherein the MCT oil is selected from coconut oil, palm kernel oil, an enriched form of coconut oil or palm kernel oil, a fractionated form of coconut oil or palm kernel and combinations thereof, and the LCT oil is selected from soybean oil, sunflower oil, olive oil, palm oil, coconut oil, canola oil, peanut oil, cottonseed oil, corn oil, safflower oil, sesame oil, grape seed oil, hemp seed oil, and flaxseed oil, and combinations thereof, the one or more cosurfactants if present are selected from propylene glycol, ethanol, propanol, butanol and glycerol, and combinations thereof, and the one or more water soluble carrier solid materials are selected from one or more sugars, sugar alcohols, and polysaccharides, and combinations thereof, wherein the one or more sugars, sugar alcohols, and polysaccharides are selected from sucrose, glucose, fructose, galactose, maltose, lactose, mannose, ribose, rhamnose, trehalose, xylose, sorbitol, lactitol, maltitol, xylitol, mannitol, erythritol, ribitol, galactitol, fucitol, iditol, inositol, lactitol and maltodextrin, and combinations thereof. In an embodiment, the polysorbate is polysorbate 80.

In an embodiment, the one or more cannabinoids, and/or a cannabinoid extract is selected from CBD, THC, and a cannabinoid distillate and combinations thereof, the one or more surfactants are selected from polysorbates, polyoxyethylene hydrogenated castor oils, and D-α-Tocopherol polyethylene glycol 1000 succinate (Vitamin E TPGS), and combinations thereof, the one or more oils are selected from medium chain triglyceride (MCT) oils and long chain triglycerides (LCT) oils, and combinations thereof, wherein the MCT oil is a fractionated form of coconut oil, and the LCT oil is sunflower oil, the one or more cosurfactants if present is propylene thereof, and the one or more water soluble carrier solid materials are sugar alcohols selected from sorbitol and mannitol. In an embodiment, the polysorbate is polysorbate 80.

In an embodiment, the one or more cannabinoids, and/or a cannabinoid extract is selected from CBD, THC, and a cannabinoid distillate and combinations thereof, the one or more surfactants are selected from polysorbates, polyoxyethylene hydrogenated castor oils, and D-α-Tocopherol polyethylene glycol 1000 succinate (Vitamin E TPGS), and combinations, the one or more oils are selected from medium chain triglyceride (MCT) oils and long chain triglycerides (LCT) oils and combinations thereof, wherein the MCT oil is fractionated coconut oil, and the LCT oil is sunflower oil, the one or more cosurfactants if present is propylene glycol, and the one or more water soluble carrier solid materials is sorbitol. In an embodiment, the polysorbate is polysorbate 80.

In an embodiment, the liquid and solid emulsifying formulations of the application further comprises other conventional acceptable ingredients known to be used in oral delivery formulations. Conventional procedures and ingredients for the selection and preparation of suitable pharmaceutical compositions are described, for example, in Remington's Pharmaceutical Sciences (2000—20th edition) and in The United States Pharmacopeia: The National Formulary (USP 24 NF19) published in 1999.

In an embodiment, the liquid and solid emulsifying formulations of the application further comprises one or more antioxidants and/or free-radical scavengers. In an embodiment, the one or more antioxidants and/or free-radical scavengers are selected from sodium sulphate, sodium metabisulphite, ascorbic acid, sodium formaldehyde sulphoxylate, ascorbyl palmitate, butylated hydroxytoluene (BHT), butylated hydroxyanisole (BHA), propyl gallate, and alpha-tocopherol, and combinations thereof.

In an embodiment, the liquid and solid emulsifying formulations of the application further comprises one or more sweeteners. In an embodiment, the one or more sweeteners are food additives permitted by a regulatory body. In an embodiment, the sweetener is advantame, acesulfame potassium, aspartame, encapsulated aspartame, calcium saccharin, erythritol, hydrogenated starch hydrolysates, isomalt, lactitol, maltitol, mannitol, monk fruit extract, neotame, potassium saccharin, sorbitol, saccharin, sodium saccharin, steviol glycosides, sucralose, thaumatin or xylitol. In an embodiment, the liquid and solid emulsifying formulations of the application further comprises one or more flavors. In an embodiment, the one or more flavors are fruit flavor, tea flavor, coffee flavor, dairy flavor, roasted flavor, or smoke flavor, bakery flavor, confectionary flavor, meat flavor, herbal flavor.

In an embodiment, the emulsifying formulations of the application are as described in examples 1 to 12.

In an embodiment, the liquid and solid emulsifying formulations of the application are suitably prepared into compositions including food products, beverage products, nutraceuticals, additives (for example, to food products, beverage products, and nutraceuticals), medicines, and/or pharmaceutical compositions. Accordingly, the present application also includes a composition comprising the liquid and solid emulsifying formulations of the application.

In an embodiment, the composition is a non-aqueous composition. In an embodiment, the composition is an aqueous composition. In an embodiment, the composition is beverage product. In an embodiment, the composition is an edible consumable, a nutraceutical, a medicine, or pharmaceutical composition. In an embodiment, the edible consumable is a food product.

Accordingly, the present application also includes a liquid beverage product comprising the emulsifying formulations of the applications and an aqueous medium. In an embodiment, the emulsifying formulations of the application self-emulsify upon contact with aqueous medium to produce an emulsion. In an embodiment, the emulsion comprises a plurality of droplets.

In an embodiment, the aqueous medium is water, milk, tea, coffee, juice, caffeinated beverages, herbal tea, energy drink, non-alcoholic beverages, alcoholic beverages, nitrogenated liquids or carbonated liquids. In an embodiment, the water is distilled water, alkaline water, purified water, mineral water, coconut water, sparkling water, and flavored water. In an embodiment, the juice is selected from fruit juice, synthetic fruit juice, natural vegetable juice, and synthetic vegetable juice.

The present application also includes a food product comprising the emulsifying formulations of the application. In an embodiment, the food product is selected from chewing or bubble gums, mints, suckers, jawbreakers, lozenges, hard candies, gummy candies, taffies, chocolates, muffins, brownies, cookies, crackers, granola or meal replacement bars, smokeless inhalation powders, honey, syrup, spreads, and dissolving strips.

In an embodiment, the emulsifying formulations of the application are for use as an additive for food product or a beverage product.

Accordingly, the present application also includes an additive for a food or beverage product wherein the additive comprises the emulsifying formulations of the application.

The present application also includes the emulsifying formulations of the application for use an additive for a food or beverage product. In an embodiment, the additive is fora beverage product. Therefore, in an embodiment, the present application also includes an additive for a beverage product wherein the additive comprises the emulsifying formulations of the application. In an embodiment, the beverage product is or comprises an aqueous medium. In an embodiment, the aqueous medium is as described above. In an embodiment, the additive is added to the aqueous medium prior to ingestion.

The dilution ratio of additive to aqueous medium will depend upon the composition of the additive and the selection of aqueous medium.

In an embodiment, the additive self-emulsifies upon contact with aqueous medium to produce an emulsion. In an embodiment, the emulsion comprises a plurality of droplets. In an embodiment, the additive self-emulsifies spontaneously or under mild agitation (such as shaking, mixing or stirring).

In an embodiment, the emulsion is clear and/or transparent. In an embodiment, the emulsion is a nanoemulsion.

The present application also includes a beverage product comprising an additive wherein the additive comprises a liquid or solid emulsifying formulation of the application.

In an embodiment, the additive may be added to any beverage product suitable for consumption by a subject. In an embodiment, the additive may be added to any edible consumable suitable for consumption by a subject.

In an embodiment, the additive is for a food product. In an embodiment, the food product is as described above. In an embodiment, the additive is be added to the food product before the cooking process. In an embodiment, the additives to be added to the food product during the cooking process. In an embodiment, additive is to be added to the food product after the cooking process. In an embodiment, the processing involves heating. In an embodiment, the food product is cooked.

The present application also includes a food product comprising an additive wherein the additive comprises an emulsifying formulation of the application.

The present application also includes a composition comprising an emulsifying formulation of the application and a carrier. The emulsifying formulation of the applications are suitably formulated into pharmaceutical compositions for administration to subjects in a biologically compatible form suitable for administration in vivo. The present application further includes a pharmaceutical composition comprising an emulsifying formulation of the application and a pharmaceutical carrier. In an embodiment, the composition or pharmaceutical formulation is a nutraceutical.

In an embodiment, the emulsifying formulations of the application may include a second active ingredient. In an embodiment, the second active agent is selected from one or more of terpene, terpene extract, an anti-insomnia, an anti-tussive, an opioid analgesic, a decongestant, a non-opioid analgesic, anti-inflammatory drug, anti-migraine drug, an anti-emetic, an anti-histamine, a proton pump inhibitors (PPI), a H2antagonist/H2 blocker, a tranquilizer, an anti-convulsant, a hypnotic, a muscle relaxant, an anti-psychotic, an anti-diarrheal, an Attention Deficit and Hyperactivity Disorder (ADHD) drug, an anti-Parkinson disease drug, a benzodiazepine, a benzodiazepine antagonist, a barbiturate, a barbiturate antagonist, a stimulant, a stimulant antagonist, an antidepressant, a nutraceutical, nicotine, a BCS Class II active ingredient, and a BCS Class IV active ingredient, and combinations thereof.

In an embodiment, the subject is a mammal. In an embodiment, the subject is human. In an embodiment, the subject is non-human mammal such as a feline or canine.

The present application also includes a kit comprising an emulsifying formulation of the application and a beverage product. In an embodiment, the emulsifying formulation of the application is an additive for a beverage product. In an embodiment, the emulsifying formulation of the application and the beverage product are in separate containers. In an embodiment, the beverage product is or comprises an aqueous medium. In an embodiment, the aqueous medium is as described above.

The present application also includes a kit for oral administration, the kit comprising the emulsifying formulations of the application and optionally instructions for use.

In an embodiment, the instructions are for use to a subject.

In an embodiment, the kit further comprises a second active ingredient.

In an embodiment, the emulsifying formulations are self-emulsifying formulations. In an embodiment, the emulsifying formulations are self-microemulsifying formulations. In an embodiment, the emulsifying formulations are self-nanoemulsifying formulations.

III. Methods and Uses

The emulsifying formulations of the application, for example, are for use in compositions including food products and beverage products, nutraceuticals, medicines, and pharmaceutical formulations. Accordingly, the present application includes a method of infusing a composition with one or more cannabinoids or a cannabinoid extract comprising adding an emulsifying formulation of the application to the composition.

In an embodiment, the composition is beverage product. In an embodiment, the composition is an edible consumable, a nutraceutical, a medicine, or pharmaceutical composition. In an embodiment, the edible consumable is a food product.

In an embodiment, the emulsifying formulations of the application may, for example, be useful as an additive in an edible consumable. Accordingly, the present application also includes a use of the emulsifying formulations of the application as an additive in an edible consumable. In an embodiment, the edible consumable is a food or a beverage. In an embodiment, the edible consumable is a beverage. The additive may be added to any edible consumable suitable for consumption by a subject

The present application further comprises a method of oral administration of one or more cannabinoids, and/or a cannabinoid extract comprising administering an amount of the emulsifying formulation of the present application to a subject. The present application further includes a use of an emulsifying formulations of the present application for orally administering one or more cannabinoids, and/or a cannabinoid extract, a use of and emulsifying formulations of the present application for preparation of a medicament for orally administering one or more cannabinoids, and/or a cannabinoid extract, as well as a formulation of the present application for orally administering one or more cannabinoids, and/or a cannabinoid extract.

In an embodiment, the emulsifying formulations of the application exhibits an improved oral bioavailability of the one or more cannabinoids, and/or a cannabinoid extract compared to the one or more cannabinoids, and/or a cannabinoid extract dissolved in an organic solvent or mixture of solvents.

The present application further comprises a method of improving the oral bioavailability of one or more cannabinoids, and/or a cannabinoid extract comprising administering an amount of the emulsifying formulation of the present application to a subject. The present application further includes a use of an emulsifying formulations of the present application for improving the oral bioavailability of one or more cannabinoids, and/or a cannabinoid extract, a use of and emulsifying formulations of the present application for preparation of a medicament for improving the oral bioavailability of one or more cannabinoids, and/or a cannabinoid extract, as well as an emulsifying formulation of the present application for improving the oral bioavailability of one or more cannabinoids, and/or a cannabinoid extract.

In an embodiment, the emulsifying formulations of the application exhibit an improved stability compared to the one or more cannabinoids, and/or a cannabinoid extract dissolved in an organic solvent or mixture of solvents.

The present application further comprises a method of improving the stability of one or more cannabinoids, and/or a cannabinoid extract comprising administering an amount of the emulsifying formulation of the present application to a subject. The present application further includes a use of an emulsifying formulations of the present application for improving the stability of one or more cannabinoids, and/or a cannabinoid extract, a use of and emulsifying formulations of the present application for preparation of a medicament for improving the stability of one or more cannabinoids, and/or a cannabinoid extract, as well as an emulsifying formulation of the present application for improving the stability of one or more cannabinoids, and/or a cannabinoid extract.

The emulsifying formulations of the application may, for example, be useful for the treatment of various diseases, disorders or conditions that are treatable by one or more cannabinoids, and/or a cannabinoid extract;

Therefore, the present application also includes a method for treating or preventing diseases, disorders or conditions treatable by one or more cannabinoids, and/or a cannabinoid extract, the method comprising administering an effective amount of the emulsifying formulation of the present application to a subject in need thereof. The present application further includes a use of an emulsifying formulations of the present application for treating diseases, disorders or conditions treatable by one or more cannabinoids, and/or a cannabinoid extract, a use of and emulsifying formulations of the present application for preparation of a medicament for treating diseases, disorders or conditions treatable by one or more cannabinoids, and/or a cannabinoid extract, as well as a formulation of the present application for use to treat diseases, disorders or conditions treatable by one or more cannabinoids, and/or a cannabinoid extract.

In an embodiment, the diseases, disorders or conditions treatable by one or more cannabinoids, and/or a cannabinoid extract are selected from intractable cancer pain, neuropathic pain, chronic pain, postoperative pain, rheumatoid arthritis, multiple sclerosis and spasticity, fibromialgia, inflammation, gastrointestinal disorders (for example, nausea and vomiting, motility disorders), acute schizophrenia, cancer, tics and behavioral problems experienced by patients with tourette's syndrome, Parkinson's disease, Huntington's disease, diabetes and diabetic complications, cerebrovascular disorders and glaucoma.

In an embodiment, the diseases, disorders or conditions treatable by one or more cannabinoids, and/or a cannabinoid extract are selected from nausea, vomiting, appetite, stress, anxiety, inflammation, pain, cancer and a neurologic disease, disorder or condition.

In an embodiment, the subject is a mammal. In an embodiment, the subject is human. In an embodiment, the subject is non-human mammal.

Treatment methods comprise administering to a subject a formulation of the application that comprises a therapeutically effective amount of one or more cannabinoids and optionally consist of a single administration, or alternatively comprise a series of administrations. In an embodiment, the formulations of the application may be administered at least once a week. In an embodiment, the formulations may be administered to the subject from about one time per three weeks, or about one time per week to about once daily for a given treatment. In another embodiment, the formulations are administered 2, 3, 4, 5 or 6 times daily. The length of the treatment period depends on a variety of factors, such as the severity of the disease, disorder or condition, the age of the subject, the concentration and/or the activity of the formulations of the application, and/or combinations thereof. It will also be appreciated that the effective dosage of the formulations used for the treatment may increase or decrease over the course of a particular treatment regime. Changes in dosage may result and become apparent by standard diagnostic assays known in the art. In some instances, chronic administration may be required. For example, the formulations are administered to the subject in an amount and for duration sufficient to treat the patient.

Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

IV. Methods of Preparation

The present application includes a method of preparing an liquid emulsifying formulation comprising:

combining one or more cannabinoids and/or a cannabinoid extract, one or more surfactants and optionally one or more co-surfactants; under conditions to form the liquid emulsifying formulation.

The present application also includes a method of preparing a liquid emulsifying formulation comprising:

a) mixing one or more cannabinoids and/or a cannabinoid extract and one or more oils under conditions for forming an oil phase;

b) optionally, warming the oil phase;

c) combining one or more surfactants and optionally one or more co-surfactants with the oil phase under conditions for forming the liquid emulsifying formulation.

In an embodiment, the conditions for producing the oil phase comprises mixing or stirring. In an embodiment, the conditions for producing the oil comprises warming the oil phase.

In an embodiment, the conditions for producing the liquid emulsifying formulation comprises mixing or stirring.

In an embodiment, the liquid emulsifying formulation produced as described above is converted to a solid self-emulsifying formulation using one or more water soluble carrier materials. Therefore, in an embodiment, the method further comprises

d) adsorbing the liquid emulsifying formulation onto one or more water soluble carrier solid materials under conditions to form the solid emulsifying formulation.

Accordingly, the present application also includes a method of preparing a solid emulsifying formulation comprising:

a) mixing one or more cannabinoids and/or a cannabinoid extract and one or more oils under conditions for forming an oil phase;

b) optionally, warming the oil phase;

c) combining one or more surfactants and optionally one or more co-surfactants with the oil phase under conditions for forming a liquid emulsifying formulation, and

d) adsorbing the liquid emulsifying formulation onto one or more water soluble carrier solid materials under conditions to form the solid emulsifying formulation.

In an embodiment, the liquid and solid emulsifying formulations are self-emulsifying formulations. In an embodiment, the liquid and solid emulsifying formulations are self-microemulsifying formulations. In an embodiment, the liquid and solid emulsifying formulations are self-nanoemulsifying formulations.

In an embodiment, the conditions to form the solid emulsifying formulation comprise extrusion (such as hot melt extrusion), spray congealing (spray cooling), spray drying, pan drying, freeze drying or plating. In an embodiment, the conditions to form the solid emulsifying formulations comprise spray congealing (spray cooling) with a molten water soluble carrier material.

In an embodiment, the conditions to form the solid emulsifying formulation comprise a non-aqueous process and/or solvent free process selected from extrusion (such as hot melt extrusion), spray congealing (spray cooling), spray drying, pan drying, and freeze drying.

In an embodiment, the conditions to form the solid emulsifying formulation comprise extrusion (such as hot melt extrusion). In an embodiment, the conditions to form the solid emulsifying formulation comprise melting the one or more water soluble carrier solid materials prior to the step of adsorbing.

In an embodiment, the conditions to form the solid emulsifying formulation comprise freeze-drying or spray drying. In an embodiment, the conditions to form the solid emulsifying formulation comprise spray drying. In an embodiment, the conditions to form the solid emulsifying formulation comprise spray drying an aqueous dispersion of the emulsifying formulation with the water soluble carrier material. In an embodiment, the conditions to form the solid emulsifying formulation comprise spray drying using nitrogen as the drying gas.

In an embodiment, the Applicants have found that the liquid emulsifying drug-delivery formulations can be converted into a solid emulsifying formulations that are, for example, free flowing powders, by plating the liquid emulsifying formulation onto one or more water soluble carrier solid materials. In embodiments, when the one or more water soluble carrier solid materials is sorbitol, the plating is performed in the presence of water. Plating onto sorbitol in the presence of water was found to produce solid emulsifying formulations as dry powders comprising the liquid emulsifying formulation adsorbed onto the surface and/or coated inside of the pores of the solid support in dry form, and not wet powders of soluble carrier solid material coated with the liquid emulsifying formulation.

Therefore, in an embodiment, the step of adsorbing the liquid emulsifying formulation onto one or more water soluble carrier solid materials under conditions to form the solid emulsifying formulation (step d) comprises plating the liquid emulsifying formulation onto one or more water soluble carrier solid materials under conditions to form the solid emulsifying formulation.

Accordingly, the present application also includes a method of preparing a solid emulsifying formulation comprising:

a) mixing one or more cannabinoids and/or a cannabinoid extract and one or more oils under conditions for forming an oil phase;

b) optionally, warming the oil phase;

c) combining one or more surfactants and optionally one or more co-surfactants with the oil phase under conditions for forming a liquid emulsifying formulation; and

d) plating the liquid emulsifying formulation onto one or more water soluble carrier solid materials under conditions to form the solid emulsifying formulation.

In an embodiment, the conditions to form the solid emulsifying formulation in the step of plating (step d) comprises water. It would be appreciated by a person skilled in the art that the water may be necessary in the conversion of the liquid emulsifying formulation, to a solid emulsifying formulation in the plating process.

In an embodiment, the water is added as an ingredient (e.g., exogenously), the water is present in the raw materials, for example, the one or more cannabinoids and/or a cannabinoid extract, the one or more oils and/or the one or more water soluble solid carrier materials, or the water is absorbed by the raw materials from the moisture in the air. In an embodiment, the water is present in the raw materials or is absorbed by the raw material from the moisture in the air.

In an embodiment, the one or more water soluble solid carrier materials is sorbitol, and the conditions to form the solid emulsifying formulation in the step of plating comprises water. In an embodiment, the water is present in the sorbital raw material. In an embodiment, the water is absorbed from the moisture in the air.

In an embodiment, the conditions to form the solid emulsifying formulation in the step of plating comprise warming the (liquid) emulsifying formulation. In an embodiment, the conditions to form the solid emulsifying formulation in the step of plating comprise warming the (liquid) emulsifying formulation and mixing the warmed emulsifying formulation with the one or more water soluble solid carrier materials. In an embodiment, the mixing comprises spraying the emulsifying formulation, optionally warmed emulsifying formulation, onto the one or more water soluble carrier solid materials with continuous stirring and/or blending. In an embodiment, the mixing is performed with a planetary mixer, granulator, roller miller, blender, or compactor. In an embodiment, the conditions to form the solid emulsifying formulation optionally further comprise an inert atmosphere. In an embodiment, the inert atmosphere is a nitrogen gas atmosphere.

In an embodiment, the solid emulsifying formulation is dried. In an embodiment, the solid emulsifying formulation is dried by dry mixing. In an embodiment, the solid emulsifying formulation is dried by heating. In an embodiment, the solid emulsifying formulation is dried by heating in an oven. In an embodiment, the solid emulsifying formulation is dried by air drying. In an embodiment, the solid emulsifying formulation is dried by fluid bed drying, drum drying, vacuum drying, or freeze drying.

In an embodiment, the solid emulsifying formulation is a powder. In an embodiment, the solid emulsifying formulation is free-flowing powder.

In an embodiment, the solid emulsifying formulation is micronized or pulverized. In an embodiment, the solid emulsifying formulation is micronized or pulverized by milling, bashing or grinding. In an embodiment, the solid emulsifying formulation is micronized using supercritical fluids.

In an embodiment, the solid emulsifying formulation is micronized or pulverized to a particle size of less than 1000 μm, less than 800 μm, less than 750 μm, less than 500 μm, less than 400 μm, less than 300 μm, less than 200 μm, less than 100 μm, less than 75 μm, less than 50 μm, or less than 25 μm. In an embodiment, the solid emulsifying formulation is micronized or pulverized to a particle size of about 10 μm to about 1000 μm, about 50 μm to about 1000 μm, about 50 μm to about 750 μm, about 50 μm to about 500 μm, about 50 μm to about 400 μm, about 50 μm to about 300 μm, about 100 μm about 500 μm, or about 200 μm to about 500 μm. In an embodiment, the solid emulsifying formulation is micronized or pulverized to a particle size of about 10 μm to about 500 μm, about 50 μm to about 500 μm, about 50 μm to about 300 μm, or about 200 μm to about 500 μm.

In an embodiment, the the weight ratio of the one or more oils to the one or more cannabinoids, and/or the cannabinoid extract is from about 1:1 to about 10:1, the weight ratio of the one or more surfactants to the one or more cannabinoids, and/or the cannabinoid extract is from about 3:1 to about 50:1 and if present, the weight ratio of the one or more co-surfactants to the one or more surfactants is from about 1:10 to about 1:1:

In an embodiment, the weight ratio of the one or more oils to the one or more cannabinoids, and/or the cannabinoid extract is from about 1:1 to about 4:1, the weight ratio of the one or more surfactants to the one or more cannabinoids, and/or the cannabinoid extract is from about 3:1 to about 20:1 and if present, the weight ratio of the one or more co-surfactants to the one or more surfactants is from about 1:10 to about 1:5

In an embodiment, the weight ratio of the one or more oils to the one or more cannabinoids, and/or the cannabinoid extract from about 2:1 to about 3:1, the weight ratio of the one or more surfactants to the one or more cannabinoids, and/or the cannabinoid extract is from about 10:1 to about 15:1, and the weight ratio of the one or more co-surfactants to the one or more surfactants is from about 1:10 to about 1:5.

In an embodiment, the one or more cannabinoids, and/or a cannabinoid extract is selected from CBD, THC, and a cannabinoid distillate and combinations thereof.

In an embodiment, the one or more oils are a combination of a MCT oil and a LCT oil. In an embodiment, the one or more oils is a MCT oil.

In an embodiment, an oil comprising MCT (MCT oil) is selected from coconut oil, palm kernel oil, enriched form of coconut oil or palm kernel oil, a fractionated form of coconut oil or palm kernel and combinations thereof. In an embodiment, the oil comprising an MCT (MCT oil) is coconut oil or a fractionated form thereof. In an embodiment, the oil comprising an MCT (MCT oil) is fractionated coconut oil.

In an embodiment, the LCT oil is selected from soybean oil, sunflower oil, olive oil, palm oil, coconut oil, canola oil, peanut oil, cottonseed oil, corn oil, safflower oil, sesame oil, grape seed oil, hemp seed oil, and flaxseed oil, and combinations thereof.

In an embodiment, the one or more oils are a combination of fractionated coconut oil and sunflower oil.

In an embodiment, the one or more surfactants is selected from polysorbates, polyoxyethylene hydrogenated castor oils, and D-α-Tocopherol polyethylene glycol 1000 succinate (Vitamin E TPGS), and combinations thereof. In an embodiment, the polysorbate is polyethylene glycol sorbitan monooleate (polysorbate 80).

In embodiment, the co-surfactant is propylene glycol, ethanol, propanol, butanol or glycerol, or combinations thereof. In embodiment, the co-surfactant is propylene glycol.

In an embodiment, the one or more water soluble solid carrier materials are selected from one or more sugars, sugar alcohols, and polysaccharides, or combinations thereof. In an embodiment, the one or more water soluble solid carrier materials are selected from one or more sugars and sugar alcohols, and combinations thereof. In an embodiment, the polysaccharide is maltodextrin. In an embodiment, the one or more sugar alcohol is selected from sorbitol, lactitol, maltitol, xylitol, mannitol, erythritol, ribitol, galactitol, fucitol, iditol, inositol, and lactitol, and combinations thereof. In an embodiment, the one or more water soluble solid carrier materials are one ore more sugar alcohols. In an embodiment, the one or more sugar alcohols are selected from sorbitol and mannitol and combinations thereof. In an embodiment, the sugar alcohol is sorbitol.

In an embodiment, the one or more surfactants are selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof, the one or more oils are selected from medium chain triglyceride (MCT) oils and long chain triglycerides (LCT) oils, and combinations thereof, the one or more cosurfactants if present are selected from propylene glycol, ethanol, propanol, butanol and glycerol, and combinations thereof, and the one or more water soluble carrier solid materials are selected from one or more sugars, sugar alcohols, and polysaccharides, and combinations thereof. In an embodiment, the polysorbate is polysorbate 80.

In an embodiment, the one or more surfactants are selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof, the one or more oils are selected from medium chain triglyceride (MCT) oils and long chain triglycerides (LCT) oils, and combinations thereof, wherein the MCT oil is selected from coconut oil, palm kernel oil, an enriched form of coconut oil or palm kernel oil, a fractionated form of coconut oil or palm kernel and combinations thereof, and the LCT oil is selected from soybean oil, sunflower oil, olive oil, palm oil, coconut oil, canola oil, peanut oil, cottonseed oil, corn oil, safflower oil, sesame oil, grape seed oil, hemp seed oil, and flaxseed oil, and combinations thereof, the one or more cosurfactants if present are selected from propylene glycol, ethanol, propanol, butanol and glycerol, and combinations thereof, and the one or more water soluble carrier solid materials are selected from one or more sugars, sugar alcohols, and polysaccharides, and combinations thereof, wherein the one or more sugars, sugar alcohols, and polysaccharides are selected from sucrose, glucose, fructose, galactose, maltose, lactose, mannose, ribose, rhamnose, trehalose, xylose, sorbitol, lactitol, maltitol, xylitol, mannitol, erythritol, ribitol, galactitol, fucitol, iditol, inositol, lactitol and maltodextrin, and combinations thereof. In an embodiment, the polysorbate is polysorbate 80.

In an embodiment, the one or more cannabinoids, and/or a cannabinoid extract is selected from CBD, THC, and a cannabinoid distillate and combinations thereof, the one or more surfactants are selected from polysorbates, polyoxyethylene hydrogenated castor oils, and D-α-Tocopherol polyethylene glycol 1000 succinate (Vitamin E TPGS), and combinations thereof, the one or more oils are selected from medium chain triglyceride (MCT) oils and long chain triglycerides (LCT) oils, and combinations thereof, wherein the MCT oil is a fractionated form of coconut oil, and the LCT oil is sunflower oil, the one or more cosurfactants if present is propylene thereof, and the one or more water soluble carrier solid materials are sugar alcohols selected from sorbitol and mannitol. In an embodiment, the polysorbate is polysorbate 80.

In an embodiment, the one or more cannabinoids, and/or a cannabinoid extract is selected from CBD, THC, and a cannabinoid distillate and combinations thereof, the one or more surfactants are selected from polysorbates, polyoxyethylene hydrogenated castor oils, and D-α-Tocopherol polyethylene glycol 1000 succinate (Vitamin E TPGS), and combinations, the one or more oils are selected from medium chain triglyceride (MCT) oils and long chain triglycerides (LCT) oils and combinations thereof, wherein the MCT oil is fractionated coconut oil, and the LCT oil is sunflower oil, the one or more cosurfactants if present is propylene glycol, and the one or more water soluble carrier solid materials is sorbitol. In an embodiment, the polysorbate is polysorbate 80.

In an embodiment, the method further comprises adding other conventional acceptable ingredients at any one of the method steps. In an embodiment, the other conventional acceptable ingredients comprises one or more antioxidants and/or free-radical scavengers. In an embodiment, the one or more antioxidants are selected from citric acid, citric acid esters of mono- and diglycerides, L-cysteine, L-cysteine hydrochloride, sodium sulphate, sodium metabisulphite, ascorbic acid, sodium formaldehyde sulphoxylate, ascorbyl palmitate, ascorbyl stearate, butylated hydroxytoluene (BHT), butylated hydroxyanisole (BHA), propyl gallate, and alpha-tocopherol, and rosemary extract, and combinations thereof. In an embodiment, the other conventional acceptable ingredients comprises a sweetener. In an embodiment, the other conventional acceptable ingredients comprises a flavor. In an embodiment, the flavor is fruit flavor, tea flavor, coffee flavor, dairy flavor, roasted flavor, smoke flavor, and combinations thereof.

In an embodiment, the method further comprises adding a second active ingredient at any one of the method steps.

The present application includes emulsifying formulations prepared by any one of the methods of the application described above.

The liquid and solid emulsifying formulation of the application can be suitably prepared into compositions including food products, beverage products, nutraceuticals, additives (for example, to food products, beverage products, and nutraceuticals), medicines, and/or pharmaceutical compositions.

Accordingly, the present application also includes a method of preparing a cannabinoid beverage product comprising mixing the liquid or solid emulsifying formulations of the application with a beverage product comprising an aqueous medium. In an embodiment, beverage product is or comprises the aqueous medium. In an embodiment, the aqueous medium is as described above.

In an embodiment, the liquid and solid emulsifying formulations of the application self-emulsify emulsifies upon contact with an aqueous medium to produce a stable emulsion. In an embodiment, the emulsion comprises a plurality of droplets. In an embodiment, the droplets have an average particle size (mean diameter) of about 10 nm to about 500 nm. In an embodiment, the droplets have an average particle size of about 10 nm to about 150 nm. In an embodiment, the droplets have an average particle size of about 10 nm to about 100 nm. In an embodiment, the droplets have an average particle size of about 20 nm to about 100 nm. In an embodiment, the droplets have an average particle size of about 20 nm to about 80 nm. In an embodiment, the droplets have an average particle size of about 20 nm to about 60 nm. In an embodiment, the liquid or the solid emulsifying formulation emulsifies in an aqueous medium to produce an emulsion that is clear and/or transparent.

In an embodiment, the mixing of the liquid and solid emulsifying formulations of the application with an aqueous media does not require agitation. In an embodiment, the mixing of the liquid and solid emulsifying formulations of the application with an aqueous media requires mild agitation. In an embodiment, the mild agitation is shaking, mixing or stirring.

The following non-limiting examples are illustrative of the present application:

### EXAMPLES

### Example 1

Thoroughly mix 25 g of cannabinoid distillate and 75 g of medium chain triglyceride (MCT oil, e.g., fractionated coconut oil) to form the oil phase. Then mix 33.3 g of the oil phase with 66.7 g of polysorbate 80 at room temperature. Put 2 g of the resultant self-nanoemulsifying drug delivery system (SNEDD) into water, where it forms a transparent nanoemulsion with Z-average particle size of 35 nm upon dissolving spontaneously. No separation was observed after centrifuged for 60 min at 6000 rpm.

### Example 2

Mix 30 g of cannabinoid distillate with 35 g MCT oil (e.g., fractionated coconut oil) and 35 g sunflower oil thoroughly to form the oil phase. Then mix 25 g of the oil phase with 75 g of Vitamin E TPGS at room temperature. Put 2 g of the resultant self-nanoemulsifying drug delivery system (SNEDD) into water, where it forms a transparent nanoemulsion spontaneously with Z-average particle size at about 23 nm upon dissolving in water. No separation was observed after centrifuged for 60 min at 6000 rpm.

### Example 3

Mixing 40 g of cannabinoid distillate with 50 g MCT oil (e.g., fractionated coconut oil) and 10 g sunflower oil till complete dissolution, and then mix 40 g of the blend with 60 g of polyoxyl (40) hydrogenated castor oil thoroughly at room temperature. Put 2 g of the resultant self nano-emulsifying drug delivery system (SNEDD) into water, where it forms a transparent nanoemulsion spontaneously with z-average particle size at about 57 nm. No separation was observed after centrifuged for 60 min at 6000 rpm.

### Example 4

Thoroughly mix 25 g of cannabinoid distillate and 75 g of MCT oil (e.g., fractionated coconut oil) to form the oil phase, then mix 20 g of the oil phase with 80 g of polysorbate 80 at room temperature. Put 2 g of the resultant self nano-emulsifying drug delivery system (SNEDD) into water, where it forms a transparent nanoemulsion with Z-average particle size at about 23 nm spontaneously when dissolved in water at room temperature. No separation was observed after centrifuged for 60 min at 6000 rpm.

### Example 5

Thoroughly mix 25 g of cannabinoid distillate and 75 g of MCT oil (e.g., fractionated coconut oil) to form the oil phase, and then mix 20 g of the oil phase with 70 g of polysorbate 80 and 10 g propylene glycol (co-surfactant) at room temperature. Put 2 g of the resultant self nano-emulsifying drug delivery system (SNEDD) into water, it forms a transparent nanoemulsion with Z-average particle size at about 20 nm spontaneously when dissolved in water at room temperature. No separation was observed after centrifuged for 60 min at 6000 rpm.

### Example 6

4 g of the SNEDD from example 1 was heated to 50 deg C. and then spray onto 96 g of sorbitol powder in a blender under constant agitation with the protection of nitrogen. After spraying all SNEDD onto the surface of sorbitol powder, mix for another 5 minutes. The resultant powder is flowable, water-soluble. Put 2 gram of powder into water at room temperature, it will dissolve upon gentle agitation and form a transparent nanoemulsion with a Z-average droplet size at about 27 nm in 20 second. No separation was observed after centrifuged for 60 min at 6000 rpm.

### Example 7

3 g of the SNEDD from example 2 was heated to 50 deg C. and then sprayed onto 97 g sorbitol powder in a blender under constant agitation with the protection of nitrogen. After spraying all SNEDD onto the surface of sorbitol powder, mix the powder or anther for another 5 minutes. Put 2 gram of powder into water at room temperature, it will dissolve upon gentle agitation and form a transparent nanoemulsion with a Z-average droplet size at about 24 nm in 20 second. No separation was observed after centrifuged for 60 min at 6000 rpm.

### Example 8

5 g of the SNEDD from example 3 was heated to 50 deg C. and then sprayed onto 95 g mannitol powder in a blender under constant agitation with the protection of nitrogen. After spraying all SNEDD onto the surface of mannitol powder, mix the powder for another 5 minutes. Put 2 gram of powder into water at room temperature, it will dissolve upon gentle agitation and form a transparent nanoemulsion with a Z-average droplet size at about 54 nm in 20 second. No separation was observed after centrifuged for 60 min at 6000 rpm.

### Example 9

5 g of the SNEDD from Example 1 was mixed with 95 g of melted sorbitol at 115 deg C. in an extruder and rapidly cooled down to room temperature. The resultant products were pulverized by a powder grinder to an average particle size of about 200 to about 500 microns. Put 2 gram of powder into water at room temperature, it will dissolve upon gentle agitation and form a transparent nanoemulsion with a Z-average droplet size at about 32 nm in 20 second. No separation was observed after centrifuged for 60 min at 6000 rpm.

### Example 10

5 g of the SNEDD from Example 1 was mixed with 95 g of melted sorbitol at 115 deg C. and spray-congealing. The size of the resultant particles are have an average particle size in the range of about 50 to about 300 micron. it will dissolve upon gentle agitation and form a transparent nanoemulsion with a Z-average droplet size of about 40 nm in 20 second. No separation was observed after centrifuged for 60 min at 6000 rpm.

### Example 11

5 g of the SNEDD from Example 4 was mixed with 20 g of water and 20 g of sorbitol and then freeze-dried. The resultant products were ground into powder to a size range from about 50 to about 500 micron. It will dissolve upon gentle agitation and form a transparent nanoemulsion with a Z-average droplet size at about 30 nm in 20 second. No separation was observed after centrifuged for 60 min at 6000 rpm.

### Example 12

5 g of the SNEDD from example 5 was heated to 50 deg C. and then sprayed onto 95 g xylitol powder in a blender under constant agitation with the protection of nitrogen. After spraying all SNEDD onto the surface of xylitol powder, mix the powder for another 5 minutes. Put 2 gram of powder into water at room temperature, it will dissolve upon gentle agitation and form a transparent nanoemulsion with a Z-average droplet size at about 22 nm in 20 second. No separation was observed after centrifuged for 60 min at 6000 rpm.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A liquid emulsifying formulation comprising:
i) one or more cannabinoids, and/or a cannabinoid extract;
ii) one or more surfactants; and
iii) one or more oils,
wherein the weight ratio of the one or more surfactants to the one or more cannabinoids, and/or the cannabinoid extract is from about 1:1 to about 20:1 and the weight ratio of the one or more oils to the one or more cannabinoids, and/or the cannabinoid extract is from about 1:1 to about 10:1.

2. (canceled)

3. The liquid emulsifying formulation of claim 1, wherein the weight ratio of the one or more surfactants to the one or more cannabinoids, and/or the cannabinoid extract is from about 3:1 to about 20:1 or about 5:1 to about 20:1.

4. The liquid emulsifying formulation of claim 3, wherein the weight ratio of the one or more surfactants to the one or more cannabinoids, and/or the cannabinoid extract is from about 10:1 to about 15:1 or about 10:1 to about 20:1.

5. The liquid emulsifying formulation of claim 1, wherein the one or more surfactants is selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof.

6. (canceled)

7. (canceled)

8. The liquid emulsifying formulation of claim 1, wherein the weight ratio of the one or more oils to the one or more cannabinoids, and/or the cannabinoid extract is from about 1:1 to about 4:1.

9. The liquid emulsifying formulation of claim 1, wherein the one or more oils are selected from one or more oils comprising fatty acids, monoglycerides, diglycerides, triglycerides, hydrolyzed triglycerides, propylene glycol mono esters, or propylene glycol diesters, or combinations thereof.

10. The liquid emulsifying formulation of claim 1, wherein the one or more oils are oils comprising long chain triglycerides (LCT oils) or medium chain triglycerides (MCT oils) or combinations thereof.

11. (canceled)

12. (canceled)

13. (canceled)

14. (canceled)

15. (canceled)

16. (canceled)

17. (canceled)

18. The liquid emulsifying formulation of claim 1, further comprising one or more co-surfactants, wherein the weight ratio of the one or more co-surfactants to the one or more surfactants is from about 1:10 to about 1:1 or about 1:10 to about 1:5.

19. (canceled)

20. (canceled)

21. (canceled)

22. A solid emulsifying formulation comprising:
i) one or more cannabinoids, and/or a cannabinoid extract;
ii) one or more surfactants;
iii) one or more oils, and
iv) one or more water soluble carrier solid materials,
wherein the weight ratio of the one or more surfactants to the one or more cannabinoids, and/or the cannabinoid extract is from about 1:1 to about 20:1 and the weight ratio of the one or more oils to the one or more cannabinoids, and/or the cannabinoid extract is from about 1:1 to about 10.

23. (canceled)

24. The solid emulsifying formulation of claim 22, wherein the weight ratio of the one or more surfactants to the one or more cannabinoids, and/or the cannabinoid extract is from about 3:1 to about 20:1, about 5:1 to about 20:1, about 10:1 to about 20:1 or about 10:1 to about 15:1.

25. (canceled)

26. The solid emulsifying formulation of claim 22, wherein the one or more surfactants is selected from polysorbates, polyglycerol fatty acids esters, polyoxyethylene fatty esters, sucrose fatty esters, lecithins, alkyl polyglycosides, polyoxyethylene hydrogenated castor oils, and D-α-Tocopherol polyethylene glycol 1000 succinate (Vitamin E TPGS), and combinations thereof.

27. (canceled)

28. (canceled)

29. (canceled)

30. (canceled)

31. The solid emulsifying formulation of claim 22, wherein the one or more oils are oils comprising long chain triglycerides (LCT oils) or medium chain triglycerides (MCT oils) or combinations thereof, wherein the LCT oil is selected from soybean oil, sunflower oil, olive oil, palm oil, coconut oil, canola oil, peanut oil, cottonseed oil, corn oil, safflower oil, sesame oil, grape seed oil, hemp seed oil, and flaxseed oil, and combinations thereof and the MCT oil is selected from coconut oil, palm kernel oil, enriched form of coconut oil or palm kernel oil, a fractionated form of coconut oil or palm kernel and combinations thereof.

32. (canceled)

33. (canceled)

34. (canceled)

35. (canceled)

36. (canceled)

37. (canceled)

38. The solid emulsifying formulation of claim 22, further comprising—one or more co-surfactants, wherein the weight ratio of the one or more co-surfactants to the one or more surfactants is from about 1:10 to about 1:1 or about 1:10 to about 1:5.

39. (canceled)

40. (canceled)

41. (canceled)

42. The solid emulsifying formulation of claim 22, wherein the one or more water soluble solid carrier materials are selected from one or more sugars, sugar alcohols, oligosaccharides and polysaccharides, or combinations thereof.

43. (canceled)

44. (canceled)

45. (canceled)

46. The solid emulsifying formulation of claim 22, wherein the one or more water soluble solid carrier materials are one or more sugar alcohols.

47. (canceled)

48. (canceled)

49. (canceled)

50. (canceled)

51. (canceled)

52. The emulsifying formulation of claim 1, wherein the cannabinoid extract is a cannabinoid distillate and/or wherein the one or more cannabinoids are selected from one of more of cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBT), tetrahydrocannabinol (THC), tetrahydrocannabinolic acid (THCA), tetrahydrocannabivarin (THCV) and tetrahydrocannabivarinic acid (THCVA), and combinations thereof.

53. (canceled)

54. (canceled)

55. The emulsifying formulation of claim 1, wherein the emulsifying formulation is a self-emulsifying formulation or a self-nanoemulsifying formulation.

56. (canceled)

57. (canceled)

58. (canceled)

59. (canceled)

60. (canceled)

61. A composition comprising the emulsifying formulation of claim 1, wherein the composition is a beverage product, or the composition is an edible consumable, a nutraceutical, or pharmaceutical composition.

62. (canceled)

63. (canceled)

64. (canceled)

65. (canceled)

66. (canceled)

67. (canceled)

68. (canceled)

69. (canceled)

70. (canceled)

71. (canceled)

72. A method of preparing a liquid emulsifying formulation comprising:
a) mixing one or more cannabinoids and/or a cannabinoid extract and one or more oils under conditions for forming an oil phase;
b) optionally, warming the oil phase;
c) combining one or more surfactants and optionally one or more co-surfactants with the oil phase under conditions for forming the liquid emulsifying formulation.

73. The method of claim 72 further comprising:
d) adsorbing the liquid emulsifying formulation onto one or more water soluble carrier solid materials under conditions to form a solid emulsifying formulation, wherein the conditions to form the solid emulsifying formulation comprise hot melt extrusion, spray congealing, spray drying, pan drying, freeze drying or plating.

74. (canceled)

75. (canceled)

76. (canceled)

77. (canceled)

78. (canceled)
